(19) United States
(12) Patent Application Publication
(10) Pub. No.: US 2005/0069596 A1
(43) Pub. Date: Mar. 31, 2005

(54) Compositions and methods comprising kava and anti-anxiety compounds

(76) Inventors: Robert T. Gow, Naples, FL (US); John Pierce, Thousand Oaks, CA (US); George W. Sypert, Naples, FL (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/945,106

(22) Filed: Sep. 20, 2004

Related U.S. Application Data
(63) Continuation-in-part of application No. 10/263,579, filed on Oct. 3, 2002.
(63) Continuation-in-part of application No. 10/273,943, filed on Oct. 18, 2002.
Application No. 10/273,943 is a continuation-in-part of application No. 10/263,579, filed on Oct. 3, 2002.
(63) Continuation-in-part of application No. 10/408,900, filed on Apr. 8, 2003.
(63) Continuation-in-part of application No. 10/273,981, filed on Oct. 18, 2002.
(60) Provisional application No. 60/326,928, filed on Oct. 3, 2001.
(60) Provisional application No. 60/369,889, filed on Apr. 3, 2002.

(51) Int. Cl. A61K 35/78; A61K 31/366

(52) U.S. Cl. 424/734; 514/460

(57) ABSTRACT

The present invention comprises compositions comprising combinations of kava extract compositions in combination with one or more anxiolytics and methods for treating conditions related to the various anxiety disorders comprising administering the compositions of the present invention. The invention further provides methods for enhancing the therapeutic activity of an anxiolytic comprising administering the anxiolyitc in combination with extract of Piper methysticin.

### RELATED APPLICATIONS

This application is a continuation-in-part of U.S. patent application Ser. No. 10/263,579, filed Oct. 3, 2002, which claims priority of U.S. Provisional Patent Application Nos. 60/326,928 and 60/369,889, and also is a continuation-in-part of U.S. patent application Ser. No. 10/273,943, filed Oct. 18, 2002, which is a continuation-in-part of U.S. patent application Ser. No. 10/263,579, filed Oct.3, 2002, and is also a continuation-in-part of U.S. patent application Ser. No. 10/408,900, filed Apr. 8, 2003, and U.S. patent application Ser. No. 10/273,981, filed Oct. 18, 2002. The entire contents of these applications are hereby expressly incorporated by reference.

### FIELD OF THE INVENTION

The invention relates to methods and compositions comprising kava extract compositions in combination with compounds that provide anxiety relief to humans or animals. Such compositions can comprise kavalactone compounds in ratios that are not found in native plant materials in combination with anxiolytics such as benzodiazepine and nonbenzodiazepines. Such compositions are useful for treating anxiety disorders at lower than currently used dosages, thereby reducing the incidence of side effects related to the pharmaceutical agents.

### BACKGROUND OF THE INVENTION

Anxiety is an emotional state commonly caused by perception of real or potential danger that threatens the security of the individual. Every person experiences a certain amount of nervousness and appropriate apprehension when faced with stressful situations. Usually the response is transient and contains a built-in control mechanism to return the individual to a normal mental and physiological state.

When anxiety becomes excessive and out of proportion, it can produce uncomfortable and potentially incapacitating psychological and physical arousal. Some individuals experience persistent, severe anxiety symptoms and possess irrational fears that significantly impair normal daily functioning. These persons often suffer from what the American Psychiatric Association has classified as an anxiety disorder. Anxiety disorders have been classified into several categories: 1) Generalized anxiety disorder; 2) Panic disorder; 3) Agoraphobia; 4) Phobic disorders; 5) Obsessive-compulsive disorder; 6) Post-traumatic stress disorder; and 7) Acute stress disorder. Anxiety disorders are among the most frequent mental disorders encountered in clinical practice. One-fourth of the population in North America and Europe will experience at least one anxiety disorder in their lifetime. However, only 27% of such patients suffering with anxiety disorders receive professional treatment. Failure to properly diagnose and manage anxiety disorders results in negative outcomes with overuse of health care resources and increased morbidity and mortality. Individuals with anxiety disorders develop other health risks, including cardiovascular, cerebrovascular, gastrointestinal and respiratory disorders, and at significantly higher rates than the general population.

According to the National Comorbidity Survey of non-institutionalized persons aged 15 to 54 years, the annual prevalence rate for anxiety disorders averaged 17.2%, and the lifetime rate was 24.9%. The annual economic burden of anxiety disorders in the U.S. was estimated to be $42.3 billion in 1990, amounting to about $1,542 per sufferer. Essentially all anxiety disorders are associated with impaired workplace performance and lost productivity. Hence, anxiety disorders represent major health and socio-economic problems.

Generally, anxiety disorders are a group of heterogeneous illnesses that develop before the age of 30 and are more common in women and those with a family history of anxiety and depression. Anxiety sufferers often develop another anxiety disorder, such as major depression or substance abuse. Anxiety disorders are usually chronic in nature, and although symptoms wax and wane over time, patients rarely are completely free of symptoms. Most patients with anxiety disorders can be treated effectively. However, long-term treatment, generally with medication, may be required, and relapse after drug discontinuation is common.

Available evidence from biochemical and neuro-imaging studies indicate that the modulation of normal and pathologic anxiety states is related to multiple brain structures and abnormal function in several neurotransmitter systems, including norepinephrine (NE), gamma-aminobutyric acid (GABA), and serotonin (5-HT).

Benzodiazepines (BZs) are one type of medication used for the amelioration of acute anxiety attack symptoms. All BZs are effective anxiolytics. Consideration of pharmacokinetic properties and the patient's clinical situation assists the physician in the selection of the most appropriate agent. Pharmacokinetic differences vary, and the treating physician must monitor the patient's response to the initial treatment regimen. Because patients do not usually develop chemical dependency, BZ have emerged as the treatment of choice for long-term management of anxiety disorders.

Other pharmaceutical agents are used for treating anxiety-related disorders. Buspirone, or its salt, for example, buspirone hydrochloride, is an antianxiety agent that is not chemically or pharmacologically related to the benzodiazepines, barbiturates, or other sedative/anxiolytic drugs. Hydroxyzine, or its pharmaceutical salt, such as hydroxyzine hydrochloride is designated chemically as 1-(p-chlorobenzhydryl) 4-[2-(2 hydroxyethoxy)-ethyl] piperazine dihydrochloride and can also be used to treat anxiety-related conditions. Because of the high risk of adverse effects and toxicity, barbiturates, anti-psychotics, antipsychotic-antidepressant combinations, and antihistamines generally are not indicated in the treatment of chronic anxiety disorders. The BZs are more effective in treating the somatic/autonomic symptoms of anxiety disorders as opposed to the psychological symptoms (i.e., apprehension, worry), which are reduced by antidepressants. Due to the clinical complexity of anxiety disorders, a variety of clinical drug algorithms have been developed and recommended.

The most common adverse effects associated with BZ therapy involve CNS depression. This is manifested clinically as drowsiness, sedation, psychomotor impairment, and ataxia. A transient mild drowsiness is experienced commonly by individuals during the first few days of treatment, however, tolerance usually develops. Disorientation, depression, confusion, irritability, aggression, and excitement have also been reported. Impairment of memory and recall also may occur during BZ treatment. Two serious complications of BZ therapy are the potential for abuse and development of physical dependency by patients prone to such reactions.

Kava plant, a type of pepper plant also known as Piper methysticum, is generally found in Polynesia, Melanesia, and Micronesia. The kava plant contains high concentrations of active kavalactones (sometimes referred to as kavapyrones), including kavain, methysticin, yangonin, dihydromethysticin, demethoxyyangonin, and dihydrokavain. Kava has long been used as an herbal medicine for the treatment of, among others, mental and physical fatigue, tension, cognitive impairment, nervous depression, headaches, weight loss, and pain. The prized part of the kava plant is the root system because it contains the highest concentrations of the active kavalactones, although kavalactones are also found in other parts of the plant at lower concentrations.

To harvest the kavalactones from the kava plant, the root of the plant is conventionally processed to generate a consumable product. In conventional and historical methods, the root is dried and ground to a powder. This powder contains not only the kavalactones, but also plant oils, tannins, resins, and other substances. The powder can be mixed with water to form a beverage or can be packaged into capsules or other forms for oral delivery.

The effects of ingesting kava root vary from person to person. Common effects include a state of relaxation and a reduction in muscle tenseness, and it can also produce a mild state of euphoria. Kava root extracts have also been used to help sufferers of insomnia. The ratios of the various concentrations of kavalactones in the consumed kava product have impacts on the physical effects experienced by the user. There are a large number of different kava cultivars, each of which has differing ratios of the active kavalactones.

Health issues associated with consuming kava have recently arisen. There have been several reports suggesting a link between kava use and liver damage. One theory suggests that a toxic chemical pipermethystine, may be present in the stem peelings and leaves of the kava plant, but not the root. As the demand for kava increased a few years ago, companies started buying the stem peelings and leaves along with the root, leading to ingestion of pipermethystine by kava users. Another theory is that two active kalalactones, methysticin and dihydromethysticin, which contain a methylenedioxyphenyl functional group, may form intermediary compounds that can inactivate multiple P450 enzymes. P450 enzymes can interfere with the metabolism of many pharmaceuticals which, under certain circumstances, could lead to liver toxicity.

One solution to these health problems is to limit the extraction of kavalactones to only the root, thus eliminating the presence of pipermethystine. Another solution is to develop new kava cultivars with reduced amounts of methysticin and dihydromethysticin and increased kavain content. Although such cultivars exist, they are very fragile, time consuming to maintain and not assured of success.

There exists a need in the art for a method for enhancing the desired physiological and medicinal activities of anxiolytics so that their benefits are achieved at a reduced dose resulting in a reduced risk of the known side effects.

### SUMMARY OF INVENTION

The present invention comprises methods and compositions of kava extracts combined with anxiety-reducing pharmaceutical agents. Such compositions are provided for oral delivery systems in the form of tablets, capsules, lozenges, liquids, and emulsions to achieve a beneficial effect with a corresponding reduced incidence of dose-related side effects. Kavalactones provide potentiating effects for pharmaceutical agents such as benzodiazepines and barbiturates. Combinations of kavalactones with anxiolytics lead to enhancement of the desired physiological and medicinal effects of the anti-anxiety medications at reduced dosages.

An aspect of the present invention comprises methods of selective extraction of compounds from extraction compositions of kava to yield compositions having differing characteristics, such as a lower risk of liver toxicity. Another aspect of the present invention comprises compositions that are combinations of compositions of kava extracts with anxiolytics. Anxiolytics as used herein include pharmaceutical agents used to treat anxiety-related conditions, and include, but are not limited to, benzodiazepine and nonbenzodiazepine anti-anxiety agents including antidepressants, such as imipramine, paroxetine, trazodone, venlafaxine, azapirones, such as buspirone, diphenylmethane, such as hydroxyzine, and beta-blockers, such as propanolol. The term also includes derivatives and pharmaceutical salts of the pharmaceutical agents.

An aspect of the present invention comprises methods and compositions comprising kava and anxiolytics. Methods of the invention comprise methods of extraction of compounds from plant source material of Piper methysticum, or kava, methods of making pharmaceutical or nutriceutical products comprising kava extract compositions and anxiolytics, and methods of use of kava extracted products and anxiolytics, and pharmaceutical and nutriceutical products made with the compositions. Compositions of the present invention comprise extraction products of kava comprising extracted kavalactones that have altered kavalactone profiles that are not found in native plant material from which the kavalactones were extracted, combined with compositions comprising pharmaceutically active agents known for treatment of anxiety-related conditions, such as the anxiolytics disclosed herein. An embodiment of the compositions of the present invention comprises compositions comprising an extract of kava having reduced levels of methysticin and dihydromethysticin and increased levels of kavain and dihydrokavain, in combination with compositions of anxiolytics. Compositions of the present invention also comprise pharmaceutical and nutriceutical compositions such as a rapid-dissolving tablet containing a combination of extracts of kava and anxiolytics. Another aspect of the present invention comprises compositions comprising extraction products of kava combined with anxiolytics that have reduced liver toxicity.

Another aspect of the present invention comprises methods for making compositions comprising kava extract compositions combined with anxiolytics compositions that have a predetermined characteristic, such as altered kavalactone profiles compared to the native kava plant materials from which they were extracted.

The compositions of the present invention comprise kava compositions having altered kavalactone profiles combined with anxiolytics, such that the compositions have characteristics, such as kavalactone profiles that are unlike those found in the unextracted native plant material and in currently known extracted compositions, such as beverage infusions. Compositions having differing predetermined kavalactone amounts allow for the production of kava compositions combined with anxiolytics for enhancing or reducing certain physiological effects when the compositions are consumed. Methods of the present invention comprise providing treatments for anxiety-related disorders, including, but not limited to, generalized anxiety disorders, panic disorders, social anxiety disorders, and posttraumatic stress disorders. Embodiments of the compositions comprise compositions comprising kava compositions combined with anxiolytics.

Another aspect of the present invention relates to formulation of oral delivery systems having the desired clinical effects of treating or preventing anxiety-related conditions. The compositions of the present invention can be used to make a combined kava extract and anxiolytics composition product in formulations such as a paste, resin, oil or powder, beverage, liquid infusion or decoction, or a dry flowable powder. Such products are processed for many different uses, and some embodiments are made into a fast-dissolve tablet or other orally available delivery vehicle.

The kava plant material is extracted and the resulting kava compositions from the extractions have altered kavalactone profiles compared to the native kava plant material prior to extraction, and can be in the form of a paste, oil or resin, or other form suitable for use or further processing. Preferably, the extraction methods include using supercritical CO₂ extraction and solvent modifiers such as water and ethyl alcohol. The extracted compositions, having altered kavalactone profiles, can then be subjected to further processing steps. Kava extract compositions produced by such methods have predetermined characteristics, such as altered kavalactone profiles that are unlike those found in the native plant materials and the altered kavalactone profile can be tailored to meet particular considerations for the final product. The kava and anxiolytics compositions .so produced can be used alone or in combination with each other, other compounds or other extracted materials, herbal remedies, pharmaceutical agents, food, dietary supplements, or beverages. The kava and anxiolytics compositions can also be used in treatments of physiological conditions. Administration of the compositions result in reduction of the amount of anti-anxiety medication needed to be effective and to achieve the beneficial effect, thus having a corresponding reduction in the incidence of dose-related side effects. In addition, the compositions comprising the kavalactone extract compositions can be used to treat anxiety disorders such as, but not limited to, apprehension, worry, inability to relax, and muscle tension.

### DETAILED DESCRIPTION OF THE INVENTION

The present invention comprises methods and compositions of Piper methysticum or kava, particularly the root material of kava, and one or more pharmaceutical compositions for treating anxiety-related conditions such as anxiolytics. Methods of the present invention comprise making compositions comprising extracted kava compositions, which include both the materials extracted from kava and the extracted residue, combined with one or more pharmaceutical compositions for treating anxiety-related conditions such as anxiolytics. Compositions of the present invention comprise compositions resulting from extraction of kava, such as compositions of extracted kava that have ratios of alkaloid compounds or kavalactones that are not found in the native plant material prior to extraction, in combination with anxiolytics. Suitable methods and compositions of kava are disclosed in U.S. patent applications Ser. Nos. 10/273,943, 10/408,900, 10/273,981, 10/263,579 and PCT/US03/25819, PCT/US02/33385, PCT/US03/31611, PCT/US02/33384 and PCT/US02/31771, and the disclosures of each are hereby incorporated by reference in its entirety as if specifically set forth herein.

The kava extract compositions of the present invention are extracts of the native plant material of Piper methysticum, particularly the root material, though other P. methysticum plant material can be used to yield the compositions taught herein. Kava extract compositions comprise one or more kavalactones. Kavalactones contemplated by the present invention include, but are not limited to, methysticin, dihydromethysticin, kavain, dihydrokavain, yangonin, and demethoxyyangonin. The kavalactones in the compositions of the present invention have ratios of one to another that are different from those found in native plant material, and are referred to herein as altered kavalactone profiles. Individual kavalactones are present in various percentages of the total kavalactone component, making up different kavalactone distribution profiles. The compositions of the present invention have altered kavalactones that may be different from the native plant material from which it is extracted, or may be different from kavalactone profiles of known cultivars of kava.

The present invention comprises compositions comprising extracts of the plant Piper methysticum, also referred to herein as kava, combined with compositions comprising anxiolytics. Another aspect of the present invention comprises methods of use of compositions comprising combinations of kava compositions and anxiolytics compositions for treatment of anxiety-related conditions. More specifically, the present invention comprises methods and compositions of combinations of kava compositions having alkaloid profiles not found in the native plant material and compositions of anxiolytics. The present invention also comprises methods and compositions of combinations of kava compositions having kavalactone profiles, such as ratios of kavalactone compounds, not found in the native kava plant material and anxiolytic compositions, such combination compositions that have lower amounts methysticin or dihydromethysticin. As used herein, the term “kavalactone profile” shall mean the ratios of kavalactone compounds found in kava, and the relative amounts of each compound in relation to the other kavalactone compounds in the plant material or in a kava extract. The kavalactone profile refers to the amount in grams of each kavalactone compound found in kava or in a kava extract. The native plant material would have a kavalactone profile exhibiting the types and amounts of kavalactone compounds made by the plant. The native plant material is referred to herein interchangeably as kava or Piper methysticum. An altered kavalactone profile or a kavalactone profile different from that of native plant material means the ratios of the kavalactone compounds of the composition are different from the ratios found in the native plant material. For example, in an altered kavalactone profile, the amount of one or more kavalactone compounds may be different or the ratios of one or more kavalactone compounds to the total amount or to other kavalactone compounds are different from those found in native plant material. Native plant materials include plant materials that may be shredded, ground or powdered after picking and drying, but no extractions, other than incidental water or oil loss, due to the physical manipulation of the plant material are included.

Compositions of the present invention comprise compositions of kava extracts, combined with compositions of anxiolytics, in formulations such as a paste, powder, or in other forms, for use in dietary supplements. The compositions can be processed to produce consumable items, for example, by mixing it in a food product or in a capsule, or providing the paste itself for use as a dietary supplement, with sweeteners and flavors added as appropriate. Accordingly, such supplements may include, but are not limited to, compositions of kava combined with anxiolytics for oral delivery in the form of tablets, capsules, lozenges, liquids, and emulsions. A dry, flowable powder formulation is also contemplated by the present invention. Other aspects of compositions of the present invention comprise kava compositions combined with anxiolytic compositions in the form of a rapid-dissolve tablet.

The kava extract compositions of the present invention are obtained by extraction of the kava components from the root of the plant. In a kava extraction process, the rate at which various components, including but not limited to the kavalactone components, are extracted varies according to the extraction solvent and extraction conditions used. However, kava root extracts are conventionally sold based upon total kavalactone content. Hence, the focus of conventional commercial extraction techniques has been to extract as much of the kavalactone content from the root source as possible to provide for the greatest total kavalactone concentration.

Although various extraction techniques have been used to process kava root, supercritical CO₂ processes have been found to extract the largest quantity of kavalactones. Thus, conventional practice has been to focus on the use of supercritical CO₂ to extract all of the kavalactones from the root in the production of kava extracts. As can be appreciated, such bulk extracts will generally contain kavalactones in the same or nearly the same proportions as present in the source root. Because of the differing physiological effects produced by variations in the kavalactone distribution profile, producers of such kava products are limited to specific kava cultivars as source materials in order to produce a kava product that produces the desired effect.

To the extent that the distribution profile of the various kavalactones in the extract have been of concern, conventional practice is to use additional processing steps, such as high pressure liquid chromatography (HPLC), to extract individual kavalactones. For example, and not for limitation, HPLC and other chromatographic techniques, such as “flash” chromatography, have been used to bulk process kava extracts and isolate individual kavalactones. These latter processes are cumbersome to implement and require the use of machinery that is bulky and prohibitively expensive to operate. Methods for extracting and purifying kava, including supercritical CO₂ and chromatography, are discussed in PCT publication WO 00/772861 to Martin et al.

More recently, methods for processing kava root to provide an extract of kava as a paste, powder, or in other forms, and to allow the kavalactone profile to be altered during processing have been disclosed. Such methods allow for reduced levels or amounts of methysticin and dihydromethysticin and increased levels or amounts of kavain and dihydrokavain. Such methods are taught in PCT/US2003/25819, PCT/US2002/33385; PCT/US2003/31611; PCT/US2002/33384 and PCT/US2002/31771 to Gow et al, all of which are incorporated herein in their entireties. Such methods can be used to make kava compositions having altered kavalactone profile compositions and that can be produced cost-effectively using a minimal number of processing steps. Additionally, the methods of Gow et al., eliminate the need for additional processing, such as chromatography. For example, and not for limitation, the kava extract compositions of the present invention contain one or more kavalactones in an amount between 0.1 mg to 400 mg. More particularly, the one or more kavalactones present in a kava extract composition range between 1 mg to 300 mg. A specific kava extract composition comprises a total kavalactone amount of between approximately 5 mg to 250 mg.

Of the total kavalactone component of the compositions of the present invention, the percentages of individual kavalactones may vary with respect to one another. For example, and not for limitation, the combined amount of methysticin and dihydromethysticin components, by mass percentage of the total kavalactones present, is less than 30%. More particularly, the combined amount of methysticin and dihydromethysticin components, by mass percentage of the total kavalactones present, may be less than 10%-20%. Within these percentages, the ratio of methysticin to dihydromethysticin may range from 1:10 to 10:1, more particularly from 1:5 to 5:1, and even more particularly from 1:2 to 2:1. Ratios of 1:1 methysticin to dihydromethysticin are specifically contemplated by the present invention.

Also for example, and not for limitation, the combined amount of kavain and dihydrokavain components by mass percentage of the total kavalactone composition is greater than 50%. More particularly, the combined amount of kavain and dihydrokavain components by mass percentage of the total kavalactone composition may be greater than 60%-80%. Within these percentages, the ratio of kavain to dihydrokavain may range from 1:10 to 10:1, more particularly from 1:5 to 5:1, and even more particularly from 1:2 to 2:1. Ratios of 1:1 kavain to dihydrokavain are specifically contemplated by the present invention.

Also for example, and not for limitation, the combined amount of yangonin and demethoxyyangonin components by mass percentage of the total kavalactone composition is less than 20%. More particularly, the combined amount of yangonin and demethoxyyangonin components by mass percentage of the total kavalactone composition may be less than 5%-15%. Within these percentages, the ratio of yangonin and demethoxyyangonin may range from 1:10 to 10:1, more particularly from 1:5 to 5:1, and even more particularly from 1:2 to 2:1. Ratios of 1:1 yangonin and demethoxyyangonin are specifically contemplated by the present invention. As used herein, demethoxyyangonin means 2H-Pyran-2-one, 4-methoxy-6-styryl-, (E)-, CAS no. 15345-89-8, and is also known as 2H-Pyran-2-one, 4-methoxy-6-styryl-, (E)-; (E)-4-Methoxy-6-(2-phenylethenyl)-2H-pyran-2-one;2H-Pyran-2-one,4-methoxy-6- (2-phenylethenyl)-,(E)-;4-Methoxy-6-styryl-2H-pyran-2-one; Demethoxyyangonin; desmethoxyyangonin; or DMY.

One embodiment of the present invention comprises compositions wherein the kava extract composition comprises combined kavalactones in an amount between 0.1 mg to 400 mg wherein the combined amount of methysticin and dihydromethysticin components, by mass percentage of the total kavalactones present, is less than 30%, the combined amount of kavain and dihydrokavain components by mass percentage of the total kavalactone composition is greater than 50%, and the combined amount of yangonin and demethoxyyangonin components by mass percentage of the total kavalactone composition is less than 20%.

Kava extract compositions of the present invention comprise reduced levels of methysticin and dihydromethysticin and increased levels of kavain and dihydrokavain. An embodiment of the present invention comprises a kava extract composition having a kavalactone profile similar to that found in extracts of “one-day” cultivars from Vanuatu that is made from kava feedstock, or native kava plant material from a less desired cultivar of Piper methysticum. Compositions having lower amounts of methysticin and dihydromethysticin and higher amounts of kavin and dihydrokavain are contemplated by the present invention. Kava extract compositions of the present invention have a kavalactone profile that is unlike those found in native kava plant material, an altered kavalactone profile, and the profile can be tailored to meet particular end product considerations. The native kava plant material, referred to herein interchangeably as kava or Piper methysticum, has not undergone extractions to remove any components, and has a kavalactone profile exhibiting the types and amounts of kavalactone compounds made by the plant.

Embodiments of the kava extract compositions of the present invention have kavalactone amounts that have been selected to be low in the combined weight percentage of methysticin (M) and dihydromethysticin (DHM), low in the combined weight percentage of desmethoxyyangonin (DMY) and yangonin (Y), and high in the combined weight percentage of kavain (K) and dihydrokavain (DHK). Kava extract compositions can be characterized according to one or more of the following attributes: (a) a combined weight percentage of six major kavalactones methysticin (M), dihydromethysticin (DHM), yangonin (Y), desmethoxyyangonin (DMY), kavain (K), and dihydrokavain (DHK) of between about 20% to a maximum of about 90%; (b) a combined weight percentage of M and DHM between about 5-15% to about 29%; (c) a ratio of Y to DMY by weight, expressed as the logarithmic function 10*LOG₁₀(Y/DMY) in dB units, from about −1 to about 2; (d) a ratio of DHK to K by weight, expressed as the logarithmic function 10*LOG₁₀(DHK/K) in dB units, from about −4 to about 1; (e) a combined weight percentage of Y and DMY between about 5% to about 20−25%, and (f) a combined weight percentage of flavokavain A and flavokavain B of between about 0.3% to about 3%.

Embodiments (c) and (d) refer to mathematical representations of relationships between the amount of Y to DMY, and DHK to K, respectively. The data is represented in dB units, and the calculation is 10 times the log value of Y% divided by DMY%, or DHK% divided by K%. For example, if the yangonin percent is 10%, and the demthoxyyangonin percent is 16%, then 10% divided b 16% equals 0.625, and the log of .625 is −0.204. 10 times −0.204 equals −2.04dB. Such calculations can be easily represented in graphical fashion, as shown by the figures in PCT/US02/31771, incorporated herein by reference in its entirety.

The kava extract compositions of the present invention also comprise other combinations of kavalactones. In accordance with the present invention, compositions of kava extract compositions can comprise compositions comprising altered kavalactone profiles comprising combined weight percentage of the six major alpha-pyrones: methysticin (M), dihydromethysticin (DHM), yangonin (Y), desmethoxyyangonin (DMY), kavain (K), and dihydrokavain (DHK); combined weight percentage of M and DHM; combined weight percentage of DHK and K; combined weight percentage of Y and DMY; ratio of the weight percentages of Y and DMY, i.e., Y/DMY; ratio of the weight percentages of DHK and K, i.e., DHK/K; and combined weight percentages of flavokavain A and flavokavain B.

An embodiment of a kava extract composition, provided in for example, a kava paste or a dry flowable power for use in oral delivery, has one or more of the following properties: total weight percentage of M +DHM +Y +DMY +DHK +K in a range of from about 20% to about 90%; combined M+DHM weight percentage ranging from about 15% or lower to a maximum of about 29%; combined weight percentage of DHK and K in a range from about 50% to about 70%−80%; combined weight percentage of Y and DMY ranging from about 5% to about 25%; ratio of Y weight percentage to DMY weight percentage, expressed as the logarithmic function 10*LOG in dB units, ranging from about −1 to a maximum of about 2; ratio of DHK weight percentage to K weight percentage, expressed as the logarithmic function 10*LOG in dB units, ranges from about −4 to about 1; and combined weight percentages of flavokavain A and flavokavain B ranges from about 0.3% to about 3%. The value of these properties in a kava extract composition can be determined using conventional analytical techniques, such as HIPLC-UV-S (High Performance Liquid Chromotography with Ultra-Violet detection (254 nm) and Chemical Standards), or HPLC Electrospray-Mass Spectrometry.

One embodiment of a kava extract compositions comprises less than about 15% of the combination of methysticin and dehyromethysticin, less than about 8% of the combination of yangonin and desmethooxyyangonin, and greater than about 70% of the combination of kavain and dihydrokavain. Another embodiment comprises up to about 7% methysticin, up to about 5% dehyromethysticin, up to about 1 % yangonin, up to about 4% desmoxyyangonin, and greater than about 38% kavain.

For a person skilled in the art, the kalactones can be extracted from Piper methysticum, preferably the root, via a variety of different means including commonly used liquid extraction methods, including liquid extraction at atmospheric pressure. Liquid extraction may be performed by any means known in the art and include solvents such as, but not limited to, ethyl alcohol, acetone, or dimethyl ether. Alternatively, liquid extraction at atmospheric pressure may be used to extract the active kava compounds from the root source, wherein compressible gases such as, but not limited to, carbon dioxide, propane, or tetrafluoroethane may be used. When using the liquid solvents at atmospheric pressure, an apparatus such as a Soxhlet extractor will efficiently extract from the root the desired resinous material, and, thereafter, the solvent can be removed via a distillation step for later reuse, leaving behind the desired kavalactones contained in a matrix of resinous material.

In a similar fashion, kava extract can be obtained from the kava root using compressible gases such as carbon dioxide in the liquid or supercritical state, or propane in the liquid state. In this method of preparation/collection of the essential oil, the kava root material is contained within a vessel that can be pressurized and the chosen liquid (compressed gas) is passed through the vessel under pressure. In the case of carbon dioxide, the pressure ranges from 1500 psi to over 5000 psi, and in the case of the other liquefied gases, the pressure is an order of magnitude less ranging from 50 psi to 400 psi, while in both cases the temperature can range from 5° C. to 60° C. To recover the resinous material within which contains the kavalactones, the extract-laden liquid is then passed through a collection vessel, wherein the liquefied gas can be collected as a vapor, leaving behind the desired kavalactones that were originally contained in the root material. Subsequently, the gas is compressed to a liquid for reuse. An improved kavalactone profile can then be achieved by performing an additional extraction step comprising use of supercritical CO₂ as a mobile phase, combined with an adsorbent material such as that used for solid-phase extractions (SPE) and/or diatomaceous earth. Alternatively, the the kavalactones, including but not limited to methysticin, dihydromethysticin, kavain, dihydrokavain, yangonin, and demethoxyyangonin, can be purchased individually from a chemical supply company such as Chromadex and mixed in the desired proportions.

Another method to obtain different kavalactone profiles is to use the root of different varieties of the kava plant or different parts of the kava plant as initial feedstock for purposes of extraction. Alternatively, chromatographic methods as described above can be used to obtain purified forms of each of the kavalactones.

Compositions of the present invention comprise extract of Piper methysticin in combination with one or more anxiolytic. As used herein, anxiolytic may include any agent known to relieve the symptoms of anxiety or symptoms associated with anxiety-related disorders. Preferred anxiolytics include one or more of the following: benzodiazepine or nonbenzodiazepine compounds.

Benzodiazepine anxiolytics include, but are not limited to, alprazolam, chlorodiazepoxide, clonazepam, clorazepate, diazepam, halazeapam, lorazepam, and oxazepam. Nonbenzodiazepine anxiolytics include, but are not limited to, imipramine, paroxetine, trazodone, venlafaxine, buspirone, hydroxyzine, and propanolol.

The compositions of the present invention are useful in providing the physiological effects of reducing anxiety in humans or animals. Compositions of the present invention comprise extracts of kava combined with anxiolytics. Such compositions have differing amounts of kavalactones than those found in the native plant material. An aspect of the present invention comprises compositions having lowered amounts of methysticin and dihydromethysticin, and increased amounts of kavain and dihydrokavain in comparison to the levels found in native kava plant materials.

Compositions of the present invention comprise extracts of kava, combined with one or more anxiolytics, as a paste, powder, or in other forms, which can be used in dietary supplements. The extracts can be processed to produce such consumable items, for example, by mixing it in a food product or in a capsule, or providing the paste itself for use as a dietary supplement, with sweeteners and flavors added as appropriate. Accordingly, such supplements may include, but are not limited to, compositions of kava extracts combined with anxiolytic compositions, for oral delivery in the form of tablets, capsules, lozenges, liquids, and emulsions. Other aspects of compositions of the present invention comprise kava extracts combined with anxiolytic compositions in the form of a rapid-dissolve tablet.

Compositions of the present invention comprise combinations of the compositions of kava with compositions of anxiolytics. Compositions of the present invention comprise one or more compounds of kava extracts in combination with one or more anxiolytics. The compositions of kava extracts may have one or more of the altered kavalactone profiles taught herein. As used herein, the term “one or more compounds” means that at least one compound, such as kavain is intended, or that more than one compound, for example kavain and dihydrokavain, is intended. As is known in the art, the term “compound” does not mean one molecule, but multiples or moles of molecules of one or more compounds.

The present invention comprises compositions comprising a combination of one or more anxiolytics with extracts of kava, comprising kavalactones in concentrations that are different from those concentrations found in native kava plant material, or with extracts wherein the amounts of methysticin and dihydromethysticin are less than the amounts of kavain and dihydrokavain. The present invention also comprises ingestible products that comprise the compositions comprising kava extracts and anxiolytics compositions taught herein. For example, the present invention comprises compositions comprising a rapid dissolve tablet, comprising an kava extract composition having an kavalactone profile wherein the kavain and dihydrokavain compounds are in a higher concentration than methysticin and dihydromethysticin compounds and one or more anxiolytics. In another embodiment of the present invention, compositions comprise an anxiolytic in combination with kava extract compositions. The kava extract compositions may comprise different ratios of kavalactones. Kava extract compositions having 0% methysticin and 100% kavain can be combined with the one or more anxiolytics.

The present invention comprises compositions and methods for making and using such combination kava extract and anxiolytic compositions, where the compositions comprise oral delivery dosage formulations comprising the compositions taught herein. An aspect of the present invention comprises a rapid dissolve tablet, comprising a composition of one or more anxiolytics in combination with a kava extract composition, wherein the kava extract composition has an altered kavalactone profile.

Once a dry powder, comprising kava extract compositions, combined with one or more anxiolytics, is obtained, it can be used in a variety of ways such as a dietary supplement, for tableting for addition to food substances or for other uses. In a particular embodiment, the powder is mixed with other ingredients to form a tableting composition of powder which can then be formed into tablets. In a particular embodiment, the tableting powder is first wet with a solvent comprising alcohol, alcohol and water, or other suitable solvents, in an amount sufficient to form a thick doughy consistency. Suitable alcohols include, but are not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated molding system, such as described in U.S. Pat. No. 5,407,339 can be used. The tablets are then removed from the mold and dried, preferably by air-drying for at least several hours at a temperature high enough to drive off the solvent used to wet the tableting powder mixture, typically between about 70° C. to about 85° F. The tablets can then be packaged for distribution.

The kava extract compositions and anxiolytic compositions can be combined using techniques and methods that are known in the art. Such techniques include, but are not limited to, mixing, blending, stirring, including mechanical stirring, and dissolving. Anxiolytic compositions comprise one or more anxiolytic compounds, or pharmaceutical salts, and optionally other pharmaceutical agents such as dilments, buffers or emulsifiers.

Methods. and compositions of the present invention comprise compositions comprising combinations of kava extract compositions and one or more anxiolytics in the form of a paste, resin, oil, or powder. An aspect of the present invention comprises compositions of liquid preparations of kava extract compositions combined with liquid preparations of anxiolytic compositions. Liquid preparations for oral administration may take the form of, for example, solutions, syrups or suspensions, or they may be presented as a dry product for reconstitution with water or other suitable vehicles prior to administration. Such liquid preparations may be prepared by conventional means with pharmaceutically acceptable additives such as suspending agents (e.g. sorbitol syrup, methyl cellulose, or hydrogenated edible fats); emulsifying agents (e.g. lecithin or-acacia); non-aqueous vehicles (e.g. almond oil, oily esters or ethyl alcohol); preservatives (e.g. methyl or propyl p-hydroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

An aspect of the present invention comprises compositions of a dry powder extract of kava combined with a dry powder of an anxiolytic composition. Such dry powder compositions may be prepared according to methods disclosed herein and by other methods known to those skilled in the art, such as, but not limited to, spray air drying, freeze drying, vacuum drying, and refractive window drying. The combined dry powder compositions can be incorporated into a pharmaceutical carrier such as, but not limited to, tablets or capsules, or reconstituted in a beverage such as a tea.

Although the extraction techniques described herein are discussed in terms of kava and anxiolytics, it should be recognized that compositions of the present invention can also comprise, in the form of a dry flowable powder or other forms, extracts from other plants such as, but not limited to, varieties of ginseng, cherry, lettuce, Echinacia, piper betel leaf, muira puama, ginger, willow, suma, damiana, areca, mate', horny goat weed, ginkgo biloba, turmeric, garlic, puncture vine, arctic root astragalus, eucommia, gastrodia, and uncaria, or pharmaceutical or nutriceutical agents.

The present invention comprises compositions comprising combinations of kava extract compositions and anxiolytic compositions in tablet formulations, and methods for making such tablets. A tableting powder can be formed by combining between about 18% to about 60% by weight of the powdered kava extract composition and about 1% to about 40% by weight of the powdered anxiolytic composition, with between about 30% to about 80% by weight of a dry water-dispersible adsorbant such as, but not limited to, magnesium carbonate, or a dilutent, such as, but not limited to, lactose. Other dry tablet additives, such as, but not limited to, one or more of a sweetener, flavoring and/or coloring agents, a binder, such as acacia or gum arabic, a lubricant, a disintegrant, and a buffer, can also be added to the tableting powder. The dry ingredients are screened to a particle size of between about 50 to about 150 mesh. Preferably, the dry ingredients are screened to a particle size of between about 80 to 100 mesh.

A wide variety of tablet formations can be made. Preferably, the tablet has a formulation that results in a rapid dissolution or disintegration in the oral cavity. The tablet is preferably of a homogeneous composition that dissolves or disintegrates rapidly in the oral cavity to release the extract content over a period of about 2 seconds or less to about 60 seconds or more, preferably about 3 to about 45 seconds, and most preferably between about 5 to about 15 seconds.

Various rapid-dissolve tablet formulations known in the art can be used. Representative formulations are disclosed in U.S. Pat. Nos. 5,464,632; 6,106,861; 6,221,392; 5,298,261; 6,221,392; and 6,200,604; the entire contents of each are expressly incorporated by reference herein as if specifically set forth. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1% and 1.5%. Additional components include maltodextrin (Maltrin , M-500) at between 1% and 5%. These amounts are solubilized in water and used as a starting mixture to which is added a composition comprising a combination of a kava extract composition and an anxiolytic composition, or individually the kava extract composition and the anxiolytic composition, along with flavors, sweeteners, such as Sucralose or Acesulfame K, and emulsifiers such as BeFlora and BeFloraPlus which are extracts of mung bean.

A particularly preferred tableting composition or powder contains about 10% to about 60% by weight of a kava extract powder and one or more anxiolytics, and about 30% to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used. A sweetener can also be included, preferably in an amount of between about 3% to about 40% by weight depending on the desired sweetness. Preferred sweetening substances include, but are not limited to, sugar, saccharin, sodium cyclamate, aspartame, and Stevia extract, used singly or in combination, although other sweeteners could alternatively be used. Flavorings, such as mint, cinnamon, citrus (e.g., lemon or orange), can also be included, preferably in an amount between about 0.001% to about 1% by weight. If a coloring is desired, natural and/or synthetic colors can be added, preferably in an amount of between about 0.5% to about 2% by weight.

Typically, this tableting composition will maintain its form without the use of a binder. If needed, however, various binders are suitable and can be added in an amount of between about 5% to about 15% by weight, or as necessary. Any binder known to one of ordinary skill in the art may be used. Preferred binders include, but are not limited to, acacia or gum arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM.RTM. (available from R. T Vanderbilt Co., Inc. of Norwalk, Conn.), larch arabogalactan, gelatin, Kappa carrageenan, copolymers of maleic anhydride with ethylene or vinyl methyl ether.

A tablet according to this aspect of this invention typically does not require a lubricant to improve the flow of the powder for tablet manufacturing. However, if it is so desired a lubricant may be provided. Any lubricant known to one of ordinary skill in the art may be used. Preferred lubricants include, but are not limited to, talc, magnesium stearate, calcium stearate, stearic acid, hydrogenated vegetable oils, and carbowax in amounts of between about 2% to about 10% by weight.

Similarly, a disintegrant is not expected to be necessary to produce rapid dissolve tablets using the present tablet composition. However, a disintegrant can be included to increase the speed with which a resulting tablet dissolves in the mouth. Any disintegrant known to one of ordinary skill in the art may be used. If desired, between about 0.5% to about 1% by weight of a disintegrant can be added. Preferred disintegrants include, but are not limited to, starches, clays, celluloses, algins, gums, crosslinked polymers (including croscarmelose, crospovidone and sodium starch glycolate), VEEGUM.RTM. HV, agar, bentonite, natural sponge, cation exchange resins, aliginic acid, guar gum, citrus pulp, and sodium lauryl sulphate.

It is also generally considered unnecessary to buffer the tablet composition. However, a buffer may be beneficial in specific formulations. Any buffering agent known to one of ordinary skill in the art may be used. Preferred buffering agents include, but are not limited to, mono- and di-sodium phosphates and borates, basic magnesium carbonate and combinations of magnesium and aluminum hydroxide.

In a preferred implementation, the tableting powder is made by mixing in a dry powdered form the various components as described above, e.g., active ingredient (extract), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total combined weight of an kava extract composition and an anxiolytic composition in the form of a dry flowable powder in a single oral dosage is typically in the range of about 80 mg to about 600 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 1/8 inch to about 3/4 inch in diameter and about 0.2 inch to 0.08 inch in thickness, and has a weight of between about 160 mg to about 1,200 mg. In addition to disk, wafer or coin shapes, the tablet can be in the form of a cylinder, sphere, cube, or other shapes. Although the tablet is preferably homogeneous, the tablet may alternatively be comprised of regions of powdered kava extract composition and anxiolytic composition separated by non-kava and non-anxiolytic regions in periodic or non-periodic sequences, which can give the tablet a speckled appearance with different colors or shades of colors associated with the kava and anxiolytic regions and the non-kava and non-anxiolytic regions Compositions of the present invention comprise oral delivery formulations wherein the effective amount of the kavalactone composition is between 0.1 mg and 400 mg, and preferably between 5 mg and 250 mg. Compositions may also comprise kavalactone compounds wherein the combined methysticin and dihydromethysticin components, by mass percentage of the major kavalactones, are less than 30%. Other compositions comprise a kavalactone profile wherein the combined kavain and dihydrokavain components, by mass percentage of the major kavalactones, are greater than 50%. Compositions may also comprise a kavalactone profile wherein the combined yangonin and desmethoxyyangonin components, by mass percentage of the major kavalactones, are less than 20%. A preferred composition comprises a kavalactone profile wherein the combined methysticin and dihydromethysticin components, by mass percentage of the major kavalactones, are less than 30%, the combined kavain and dihydrokavain components, by mass percentage of the major kavalactones, are greater than 50%, and the combined yangonin and demethoxyyangonin components, by mass percentage of the major kavalactones, are less than 20%. The major kavalactones means the total amount of kavalactones extracted and is the combined weight of kavain, dihydrokavain, methysticin, dihydromethysticin, yangonin and desmethoxyyangonin.

One or more of the above compositions of kavalactones can be combined with the one or more anxiolytic compounds in amounts effective for reducing the condition related to anxiety. For illustration, and not for limitation, the following amounts of anti-anxiety agents may be combined with a kavalactone composition taught herein: Alprazolam, in an amount per dose between 0.01 mg and 4 mg, and preferably between 0.05 mg and 2 mg; Chlorodizepoxide in an amount per dose between 1.0 mg and 100 mg, and preferably between 3 mg and 50 mg; Clonazepam in an amount per dose between 0.01 mg and 4 mg, and preferably between 0.05 mg and 2 mg; Clorazepate in an amount per dose between 0.1 mg and 60 mg, and preferably between 0.5 mg and 30 mg; Diazepam in an amount per dose between 0.01 mg and 40 mg, and preferably between 0.1 mg and 20 mg; Halazepam in an amount per dose between 0.1 mg and 160 mg, and preferably between 1 mg and 80 mg; Lorazepam in an amount per dose between 0.01 mg and 10 mg, and preferably between 0.05 mg and 5 mg; and Oxazepam in an amount per dose between 0.5 mg and 120 mg, and preferably between 1 mg and 60 mg; Imipramine in an amount per dose between 0.1 mg and 200 mg, and preferably between 1 mg and 100 mg; Paroxetine in an amount per dose between 0.1 mg and 50 mg, and preferably between 1 mg and 30 mg; Trazodone in an amount per dose between 1 mg and 400 mg, and preferably between 10 mg and 200 mg; Venlafaxine in an amount per dose between 1 mg and 225 mg, and preferably between 5 mg and 120 mg; Buspirone in an amount per dose between 0.1 mg and 60 mg, and preferably between 1 mg and 30 mg; Hydroxyzine in an amount per dose between 1 mg and 400 mg, and preferably between 5 mg and 200 mg; and Propranolol in an amount per dose between 1 mg and 160 mg, and preferably between 5 mg and 80 mg.

Compositions of the present invention comprise oral delivery formulations wherein the amount of kavalactones combined per dose is between about 0.05 mg and about 300 mg. The amount of the combined kavalactones per dose may also be between about 1 mg and about 100 mg. Compositions of the present invention may also comprise kava compounds wherein the percentages of methysticin and dihydromethysticin are reduced with an increase of kavain and dihydrokavain. In such compositions, the amount of kavain per dose is between about 0.01 mg and about 100 mg. The amount of kavain per dose may also be between about 0.5 mg and about 10 mg.

The composition of the present invention may be administered by any means known in the art. Preferred administration includes enteral, for example oral or rectal, or parenteral administration. Particularly preferred administration is by oral means. A wide variety of oral delivery system formulations including, but not limited to, tablets, capsules, lozenges, liquids, and emulsions are contemplated by the present invention. The production of such delivery systems is readily achieved by those familiar with the art.

The present invention further comprises methods for treating anxiety disorders and conditions relating to anxiety disorders, or anxiety-related conditions. Anxiety disorders or anxiety-related conditions may include any illness, physiological or psychological, resulting in apprehension and/or nervousness. Examples of anxiety-related conditions or anxiety disorders include generalized anxiety disorders, panic disorders, agoraphobia, phobic disorders, obsessive-compulsive disorders, post-traumatic stress disorder, and acute stress disorders. Conditions relating to an anxiety disorder are included within the scope of the present invention and include chemical dependency, depression, fears, irritability, anger, nervousness, insomnia or other sleep disruptions, apprehension, worry, racing thoughts, inability to concentrate, and the like.

The combined kava and anxiolytic compositions may be administered daily, for one or more times, for effective treatment of acute or chronic conditions. Alternatively, separate kava and anxiolytic compositions may be administered together for one or more times. Such compositions may be administered as a combined composition, or as separate compositions, daily for an indefinite period. One method of the present invention comprises administering at least one time a day a composition comprising kava compounds and at least one anxiolytic. Methods also comprise administering such compositions more than one time per day, more than two times per day, more than three times per day and in a range from 1 to 15 times per day. Such administrations may be continuously, as in every day for a period of days, weeks, months or years, or may occur at specific times to treat or prevent specific conditions. For example, a person may be administered kava and anxiolyitc compositions at least once a day for years to treat anxiety-related conditions or to enhance mental focus, cognition, and sense of well being.

The methods of the present invention comprise administering an effective amount of a composition of the present invention as described above. Formulations and dosage regimens are described above, and variations thereof are within the expertise of an experienced physician. Administration includes, but is not limited to, providing an oral dose of an effective amount of kavalactone compounds and one or more of the anti-anxiety agents at least one time per day. Such compositions may be administered daily for an indefinite period of treatment of the anxiety disorders. Additionally, such compositions may be administered more than one time per day for as many days as are needed to provide relief from the condition to be treated.

The present invention comprises methods of potentiating the activity of one or more anxiolytic compounds comprising administering the compositions disclosed herein. Methods of providing dietary supplementation are contemplated. The compositions may optionally comprise vitamins, minerals and antioxidants.

Whereas this invention has been described in detail with particular reference to its most preferred embodiment, it is understood that variations and modifications can be effected within the spirit and scope of the invention, as described herein before and as defined in the appended claims. The corresponding structures, materials, acts, and equivalents of all means plus function elements, if any, in the claims below are intended to include any structure, material, or acts for performing the functions in combination with other claimed elements as specifically claimed.

### EXAMPLES

### Example 1 An example formulation of the present invention comprises the following:

| | |
|---|---|
| Extract of Piper methysticum | 90.0 mg |
| Kavalactones 75% by weight | |
| Diazepam | 2.0 mg |
| Stevioside (Extract of Stevia) | 25.0 mg |
| Carboxymethylcellulose | 50.0 mg |
| Lactose | 100.0 mg |
| Bitterness blocker | 3.0 mg |
| Vitamin C | 2.0 mg |
| Magnesium Carbonate | 28.2 mg |
| TOTAL | 300.0 mg |

The formulation can be made into any oral dosage form and administered daily to persons or animals or up to 10 times per day as needed for the physiological effects of reduced anziety, both physical and mental. For those accustomed to taking kava, more frequent administration may be needed. Optionally, the amount of kava extract or benzodiazepine can be increased in each individual dosage form, and taken as needed to maintain the level of effectiveness. CLAIMS

1. A composition, comprising, a combination of a composition comprising an extract of kava with a composition comprising one or more anxiolytics.

2. The composition of claim 1, wherein the composition comprising an extract of kava comprises one or more kavalactones.

3. The composition of claim 2, wherein the kavalactones are kavain, dehydroxykavain, methysticin, dihydromethysticin, yangonin, or demethoxyyangonin.

4. The composition of claim 2, wherein the combined weight of the kavalactones is between 0.1 mg and 400 mg.

5. The composition of claim 4, wherein the combined weight of the kavalactones is between 5 mg and 250 mg.

6. The composition of claim 2, wherein the one or more kavalactones comprises a combination of methysticin and dihydromethysticin that is less than 30% of the total kavalactones.

7. The composition of claim 2, wherein the one or more kavalactones comprises a combination of kavain and dihydrokavain that is greater than 50% of the total kavalactones.

8. The composition of claim 2, wherein the one or more kavalactones comprises a combination of yangonin and demethoxyyangonin that is less than 20% of the total kavalactones.

9. The composition of claim 2, wherein the one or more kavalactones comprises a combination of methysticin and dihydromethysticin of less than 30% of the total kavalactones, a combination of kavain and dihydrokavain of greater than 50% of the total kavalactones, and a combination of yangonin and demethoxyyangonin of less than 20% total kavalactones.

10. The composition of claim 1 wherein the anxiolytic is in an amount per dose of between 0.01 mg and 400 mg.

11. The composition of claim 10, wherein the one or more anxiolytic is alprazolam, chlorodiazepoxide, clonazepam, clorzaepate, diazepam, halazeapam, lorazepam, oxazepam, imipramine, paroxetine, trazodone, venlafaxine, buspirone, hydroxyzine, or propanolol.

12. A method for enhancing the effects an anxiolytic, comprising, administering effective amounts of a composition comprising an anxiolytic composition comprising one or more anxiolytics, and a composition comprising a kava extract, to a human or animal.

13. The method of claim 12, wherein the composition comprising an extract of kava comprises one or more of the kavalactones, kavain, dehydroxykavain, methysticin, dihydromethysticin, yangonin, or demethoxyyangonin.

14. The method of claim 12, wherein the composition comprising a kava extract comprises kavalactones in an amount between 0.1 mg and 400 mg and the composition comprising an anxiolytic composition comprises one or anxiolytics in an amount between 0.01 mg and 400 mg..

15. The method of claim 12, wherein the composition comprising a kava extract comprises a combination of methysticin and dihydromethysticin of less than 30% of the total kavalactones, a combination of kavain and dihydrokavain of greater than 50% of the total kavalactones, and a combination of yangonin and demethoxyyangonin of less than 20% total kavalactones.

16. The method of claim 12, wherein the one or more anxiolytics is alprazolam, chlorodiazepoxide, clonazepam, clorzaepate, diazepam, halazeapam, lorazepam, oxazepam, imipramine, paroxetine, trazodone, venlafaxine, buspirone, hydroxyzine, or propanolol.

17. A method for treating an anxiety disorder in a human or animal, comprising, administering to a human or animal with an anxiety disorder an effective amount of a composition comprising an anxiolytic composition comprising one or more anxiolytics, and a composition comprising a kava extract.

18. The method of claim 17, wherein the composition comprising a kava extract comprises kavalactones in an amount between 0.1 mg and 400 mg and the composition comprising an anxiolytic composition comprises one or anxiolytics in an amount between 0.01 mg and 400 mg..

19. The method of claim 17, wherein the composition comprising a kava extract comprises a combination of methysticin and dihydromethysticin of less than 30% of the total kavalactones, a combination of kavain and dihydrokavain of greater than 50% of the total kavalactones, and a combination of yangonin and demethoxyyangonin of less than 20% total kavalactones.

20. The method of claim 17, wherein the one or more anxiolytics is alprazolam, chlorodiazepoxide, clonazepam, clorzaepate, diazepam, halazeapam, lorazepam, oxazepam, imipramine, paroxetine, trazodone, venlafaxine, buspirone, hydroxyzine, or propanolol.
